(19) United States
(12) Patent Application Publication
(10) Pub. No.: US 2022/0089474 A1
(43) Pub. Date: Mar. 24, 2022

(54) BIOCOMPATIBLE GLASS SUBSTRATE WITH THROUGH ELECTRODE AND BIOCOMPATIBLE SMALL ELECTRONIC DEVICE

(71) Applicant: Schott Japan Corporation, Koka (JP)

(72) Inventors: Yutaka ONEZAWA, Koka (JP); Akira OKUNO, Koka (JP); Kazuhito MIYAWAKI, Koka (JP)

(73) Assignee: Schott Japan Corporation, Koka (JP)

(21) Appl. No.: 17/297,971

(22) PCT Filed: Nov. 18, 2019
(86) PCT No.: PCT/JP2019/045000

§ 371 Date: May 27, 2021

(30) Foreign Application Priority Data
Nov. 27, 2018 (JP) 2018-221250

(51) Int. Cl. C03C 4/00; H01L 23/15; H05K 1/11; H01L 23/498

(52) U.S. Cl. CPC C03C 4/0021; H01L 23/49827; H05K 1/113; H01L 23/15

(57) ABSTRACT

A biocompatible glass substrate with through electrodes includes a glass plate of a biocompatible glass, and through electrodes made of a biocompatible metal that are provided by penetrating the glass plate. A biocompatible electronic device using this is the biocompatible electronic device including a biocompatible glass substrate with through electrode having a glass plate of a biocompatible glass, and through electrodes made of a biocompatible metal provided by penetrating the glass plate, and an electric/electronic device sealed onto the above described glass plate and is electrically connected to the above described through electrodes, and has bumps for connection on the through electrodes of the biocompatible electronic device.

### TECHNICAL FIELD

The present invention relates to a biocompatible glass substrate with through electrode and a biocompatible small electronic device that are applied to an electric/electronic device for implant.

### BACKGROUND ART

Implant (implant) is a general term for instruments that are implanted in bodies. Implants are widely applied for medical purposes, and there are artificial roots (dental implants) that are implanted into jawbones in place of lost roots, bolts for fixing bones in treatments of fractures, rheumatism and the like, but there are also implants including an electric/electronic device that is actively driven by electric power like cardiac pacemakers, and implanted parts of cochlear implants. Among them, the present invention is applied to an implant type electric/electronic device that is used by being implanted in a body.

The implant type electronic device includes, for example, an implanted type pressure sensor for a shunt system described in Patent Literature 1. The pressure sensor stores a microchip having a pressure sensor 4, two separate substrates 1a and 1b, and other electronic components 2 and 3, in a housing having a rotating portion 7 made of titanium, and all the components are fixed into a housing 7 by a potting compound 8. A film 11 composes a membrane, a cavity portion filled with air is under the membrane, and the cavity portion is directly connected to the pressure sensor. An end portion of the housing 7 is sealed with a cap 7a, and welded. It is disclosed that the electronic components are installed on a circuit substrate 1, and a measurement signal is transmitted to a receiving unit installed outside by a sensor coil 13. An implanted type medical apparatus described in Patent Literature 2 includes a sensor such as a pressure sensor configured to be implanted in an inspection target. Once implanted, the sensor is subjected to various operating environmental conditions and the sensor is packaged in a liquid encapsulation to be protected against adverse effects under these conditions. The present invention relates to a biocompatible small electronic device and a biocompatible glass substrate with through electrode that are used in such an implant type electronic device.

As an example of the glass substrate with a through electrode, there is a method described in Patent Literature 3. Patent Literature 3 discloses an insulating substrate in which a heat resistant member is penetrated through and implanted in a glass or glass ceramics material by bringing the heat resistant member in a predetermined shape, and an insulating substrate material of glass or glass ceramics having a softening point at a lower temperature than a softening point of the heat resistant member into contact with each other, heating the insulating substrate material into a softening state and applying a weighting load, applying fusion-bonding work to a united member in which the heat resistant member is bitten into the insulation substrate material, and subjecting the united member after slow cooling to finishing work including surface polishing.

### DOCUMENT LIST

### Patent Literatures

Patent Literature 1: Japanese Patent Application Publication No. 2016-145827

Patent Literature 2: Japanese Translation of PCT International Application Publication No. 2018-516102

Patent Literature 3: Japanese Patent Application Publication No. 2007-067387

### SUMMARY OF INVENTION

### Technical Problem

Generally, an electronic device used in an implant type electronic device is required to be a particularly small shape element in order to support implanting in the relevant site while reducing a burden on a living body. In some medical applications, the implant type electronic device that is implanted is retained in an environment in a body of a subject (for example, in a vicinity of a particular organ in a body) for a certain period. During operation, the implanted electronic device is exposed to various conditions of the environment in the body for that period. The conditions have various effects on a short-term and long-term operations of the implanted electronic device. For example, environmental substances in the body such as body fluids can corrode components of the implanted electronic device. Corrosion reduces the ability to monitor biological conditions or the ability to perform medical procedures.

The present invention provides a biocompatible glass substrate with through electrode that has robustness even when placed in a harsh environment in a body, can suppress adverse effects on a living body to minimum, and is excellent in biocompatibility, and contributes to miniaturization of a biocompatible electronic device.

### Solution to Problem

According to a first aspect of the present invention, a biocompatible glass substrate with through electrode including a glass plate made of a biocompatible glass, and a through electrode made of a biocompatible metal provided by penetrating the glass plate is provided.

According to a second aspect of the present invention, a biocompatible device in which the above described biocompatible glass substrate with through electrode is applied to an electric/electronic device is provided. As the biocompatible device according to the present invention, a biocompatible device including a biocompatible glass substrate with through electrode having a glass plate made of a biocompatible glass, and a through electrode made of a biocompatible metal provided by penetrating the glass plate, and an electric/electronic device that is sealed onto the above described glass plate and has circuit wiring of the electric/electronic device electrically connected to the above described through electrode is provided. A bump for connection is included on the through electrode of the aforementioned biocompatible device.

According to a third aspect of the present invention, a manufacturing method of a biocompatible electronic device according to the present invention includes 1) a preparation step of preparing a biocompatible glass substrate wafer with through electrode including a glass plate made of a biocompatible glass and through electrodes made of a biocompatible metal provided by penetrating the glass plate, and an electric/electronic device wafer, 2) a wafer mounting step of butting and bonding desired electrodes of the biocompatible glass substrate wafer with through electrode and the electric/electronic device wafer to each other, 3) a biocompatible glass sealing step of heating the mounted wafers in a furnace to hermetically seal a contact interface of the biocompatible glass and the electric/electronic device and electrically connecting the mutual electrodes to integrally form the biocompatible glass substrate wafer with through electrode and the electric/electronic device wafer, 4) a bump forming step of forming bumps for connection onto the through electrodes of the wafer integrally formed, after the biocompatible glass sealing step, and 5) a dicing step of dicing the integrally formed wafer after bump formation, to separate the wafer into individual electric/electronic elements to make the biocompatible electronic device. In the bump forming step, instead of the bump, a different electric/electronic component from the aforementioned electric/electronic device may be mounted on the through electrode. Further, a polishing step of rounding corners of the biocompatible electronic device may be added as necessary to reduce a mechanical irritation given to a living body. Furthermore, a surface coating step to apply biocompatible coating onto a surface of the biocompatible electronic device may be added.

### Effects of Invention

Briefly explaining effects obtained by a typical one of the inventions of the present disclosure, the effects are as follows. According to one embodiment of the present disclosure, the biocompatible glass substrate with through electrode excellent in biocompatibility is provided, and contribution is made to miniaturization of a biocompatible electronic device.

### BRIEF DESCRIPTION OF DRAWINGS

FIG. 1 illustrates a perspective view of a biocompatible glass substrate 10 with a through electrode according to the present invention.

FIG. 2 illustrates a biocompatible electronic device 20 according to the present invention, (a) illustrates a plan view, (b) illustrates a front sectional view cut along line D-D in (c), and (c) illustrates a bottom view.

FIG. 3 is a flowchart illustrating a manufacturing method 30 of the biocompatible electronic device according to the present invention.

FIG. 4 is a flowchart illustrating a manufacturing method 40 of the biocompatible electronic device according to the present invention.

### DESCRIPTION OF EMBODIMENTS

A biocompatible glass substrate with through electrode according to the present invention is composed of a glass plate made of a biocompatible glass composed of only silicon, boron, calcium, sodium, phosphorus, and oxygen that are elements existing in a body, including at least one of Na₂O and CaO in a base of SiO₂ as main components, and further including at least one of B₂O₃ and P₂O₃ as necessary, and a through electrode made of a biocompatible metal that is provided by penetrating a plate surface of the glass plate and is made of any one of platinum, tantalum, tungsten, titanium, titanium alloys (for example, 90Ti-6Al-4V), Co-Cr alloys (for example, 63Co-30Cr-7Mo, 52Co-21Cr-16W-11Ni), and stainless steel (for example, 67.47Fe-18Cr-12Ni-2.5Mo-0.03C of SUS316L, 67.44Fe-18Cr-12Ni-2.5Mo-0.06C of SUS316) that are insoluble and noncorrosive in an environment in a body. It is necessary to compose at least an electrode surface of the through electrode of the aforementioned biocompatible metal, but it is preferable to compose the entire electrode of only the aforementioned biocompatible metal.

The biocompatible electronic device according to the present invention is what is made by applying the above described biocompatible glass substrate with through electrode to an electric/electronic device such as a semiconductor element to be used by being implanted into a living body. The biocompatible electronic device according to the present invention is made of a biocompatible glass substrate with through electrode having a glass plate made of a biocompatible glass composed of only silicon, boron, calcium, sodium, phosphorus, and oxygen that are elements existing in a body, including at least one of Na₂O and CaO in a base of SiO₂ as main components, and further including at least one of B₂O₃ and P₂O₃ as necessary, and a through electrode made of a biocompatible metal provided by penetrating the glass plate and is made of any one of platinum, tantalum, tungsten, titanium, titanium alloys (for example, 90Ti-6Al-4V), Co—Cr alloys (for example, 63Co-30Cr-7Mo, 52Co-21Cr-16W-11Ni), and stainless steel (for example, 67.47Fe-18Cr-12Ni-2.5Mo-0.03C of SUS316L, 67.44Fe-18Cr-12Ni-2.5Mo-0.06C of SUS316) that are insoluble and noncorrosive in an environment in a body, and an Si semiconductor element sealed onto the glass plate of the glass substrate and electrically connected to the above described through electrode, and has a cylindrical or trapezoidal or a semispherical or substantially spherical bump on the through electrode on the glass surface facing the sealed surface of the Si semiconductor element. For the bump, any material may be used, as long as the material is a soft metal made of a biocompatible metal, and the material is not limited, but a bump of gold or a gold alloy is preferable. In the above described biocompatible electronic device, a circuit surface of the Si semiconductor element is hermetically sealed and protected by the above described biocompatible glass substrate with through electrode.

In the biocompatible electronic device according to the present invention, biocompatible coating formed from an organic chemical material selected from nylon, polypropylene, polytetrafluoroethylene, a polyamide resin and the like, or an inorganic chemical material selected from calcium hydroxide phosphate, TiN, bioglass, carbon, alumina ceramics, zirconia ceramics and the like may be further applied to a surface.

The aforementioned biocompatible coating may be an organic/inorganic composite coating composed of the aforementioned organic chemical material and the aforementioned inorganic chemical material.

A manufacturing method of a biocompatible electronic device according to the present invention includes 1) a preparation step of preparing a biocompatible glass substrate wafer with through electrode having a glass plate made of a biocompatible glass composed of only silicon, boron, calcium, sodium, phosphorus, and oxygen that are elements existing in a body, including at least one of Na₂O and CaO in a base of SiO₂ as main components, and further including at least one of B₂O₃ and P₂O₃ as necessary, and through electrodes made of a biocompatible metal provided by penetrating the glass plate and are made of any one of platinum, tantalum, tungsten, titanium, and stainless steel that are insoluble and noncorrosive in an environment in a body, and an Si semiconductor wafer to be used by being implanted in a living body, 2) a wafer mounting step of butting desired electrodes of the biocompatible glass substrate wafer with through electrode and the Si semiconductor wafer to each other and bonding to the glass surface, 3) a biocompatible glass sealing step of heating the mounted wafers in a furnace to hermetically seal a contact interface of the biocompatible glass and the Si semiconductor element and electrically connecting the mutual electrodes to form a glass sealed semiconductor wafer, 4) a bump forming step of thereafter forming cylindrical or trapezoidal, or semispherical or substantially spherical bumps on the through electrodes of the biocompatible glass sealed semiconductor wafer, and 5) a dicing step of dicing the biocompatible glass sealed semiconductor wafer after bump formation to separate the biocompatible glass sealed semiconductor wafer into individual electric/electronic elements to make the biocompatible electronic device.

In the above described bump forming step, other electric/electronic elements such as active components formed of a different semiconductor element from the above described Si semiconductor element, passive components such as capacitors, resistors, coils, antennas, and various sensors, for example, or mechanism components may be mounted on the through electrodes instead of some of the bumps. For the bumps, any material may be used as long as the material is a biocompatible soft metal, and the material is not limited, but bumps of gold or a gold alloy are preferable. Further, a polishing step of rounding corners of the biocompatible electronic device may be added to reduce mechanical irritations given to a living body as necessary. For example, the polishing step can include at least any one of arbitrary polishing steps such as fire polish for rounding corners of the glass surface, chemical and mechanical polishing for rounding corners of a semiconductor end surface. Further, it may be suitable to add a coating step of applying a biocompatible coating material composed of an organic chemical material such as nylon, polypropylene, polytetrafluoroethylene, and a polyamide resin, or an inorganic chemical material such as calcium hydroxide phosphate (for example, Ca₁₀(PO₄)₆(OH)₂) of hydroxyapatite), TiN, bioglass (Na₂O—CaO—SiO₂—P₂O₃), carbon, alumina ceramics, or zirconia ceramics to a part or a whole of the biocompatible electronic device surface. As a coating method of the coating material to the surface of the biocompatible electronic device, any method may be used, and the coating method is not particularly limited, but can be thin film coating of a thickness of approximately 2 to 30 μm by, for example, Chemical Vapour Deposition (CVD).

In the aforementioned coating step, organic and inorganic composite coating may be performed by initially applying coating of an organic chemical material selected from nylon, polypropylene, polytetrafluoroethylene, a polyamide resin and the like to a surface of the biocompatible electronic device by the aforementioned CVD, and thereafter further applying coating selected from calcium hydroxide phosphate (for example, Ca₁₀(PO₄)₆(OH)₂) of hydroxyapatite), TiN, bioglass (Na₂O—CaO—SiO₂—P₂O₃), carbon, alumina ceramics, zirconia ceramics and the like.

### EXAMPLES

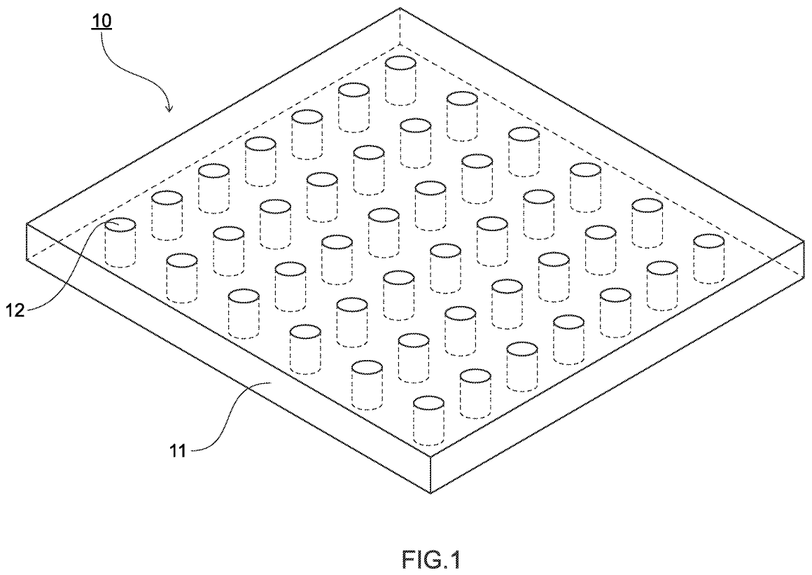

A biocompatible glass substrate 10 with a through electrode of example 1 according to the present invention is composed of a glass plate 11 formed of biocompatible glass composed of only silicon, calcium, sodium, and oxygen that are elements existing in a body and including Na₂O and CaO in a base of SiO₂, and through electrodes 12 that are provided by penetrating a plate surface of the glass plate 11 and made of platinum that is a biocompatible metal, as illustrated in FIG. 1.

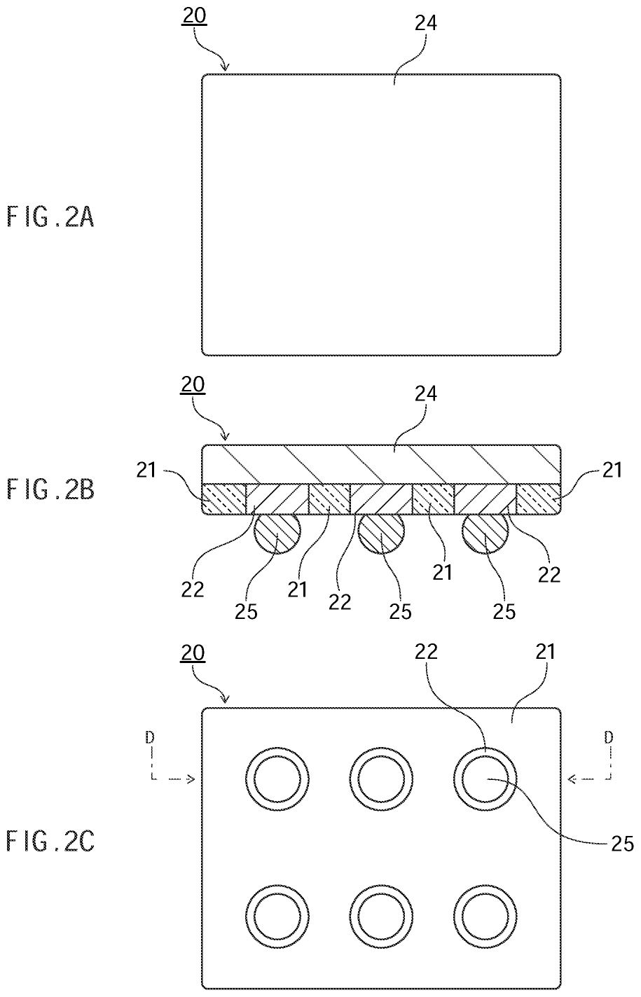

A biocompatible electronic device 20 of example 2 according to the present invention is a biocompatible electronic device in which the above described biocompatible glass substrate 10 with through electrodes is applied to an electrode material and an insulating material of the Si semiconductor element to be used by being implanted in a living body. As illustrated in FIG. 2, the biocompatible electronic device 20 has a biocompatible glass substrate 23 with through electrodes having a glass plate 21 of a biocompatible glass composed of only silicon, calcium, sodium, and oxygen that are elements existing in a body, and including Na₂O and CaO in a base of SiO₂ as main components, and through electrodes 22 made of a biocompatible metal that are provided by penetrating the glass plate 21, are insoluble and noncorrosive in an environment in a body and are composed of platinum, and an Si semiconductor element 24 sealed onto the glass plate 21 of the glass substrate 23 and electrically connected to the through electrodes 22, and has substantially spherical gold bumps 25 on the through electrodes 22 on another glass surface different from the sealed surface of the Si semiconductor element 24.

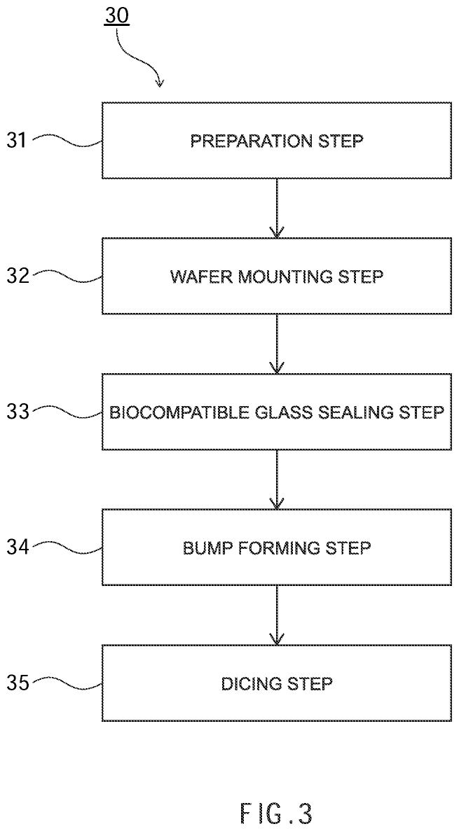

A manufacturing method 30 of a biocompatible electronic device of example 3 according to the present invention is a manufacturing method of the above described biocompatible electronic device 20. As illustrated in FIG. 3, the manufacturing method 30 of a biocompatible electronic device includes 1) a preparation step 31 of preparing a biocompatible glass substrate wafer with through electrode having a glass plate made of a biocompatible glass composed of only silicon, calcium, sodium, and oxygen that are elements existing in a body, including Na₂O and CaO in a base of SiO₂ as main components, and through electrodes made of a biocompatible metal provided by penetrating the glass plate and are made of platinum that is insoluble and noncorrosive in an environment in a body, and an Si semiconductor wafer to be used by being implanted in a living body, 2) a wafer mounting step 32 of butting desired electrodes of the biocompatible glass substrate wafer with through electrode and the Si semiconductor wafer to each other and bonding to the glass surface, 3) a biocompatible glass sealing step 33 of heating the mounted wafers in a furnace to hermetically seal a contact interface of the biocompatible glass and the Si semiconductor element and electrically connecting the mutual electrodes to form a glass sealed semiconductor wafer, 4) a bump forming step 34 of thereafter forming substantially spherical gold bumps on the through electrodes on another side of the biocompatible glass sealed semiconductor wafer, and 5) a dicing step 35 of dicing the biocompatible glass sealed semiconductor wafer after bump formation to separate the biocompatible glass sealed semiconductor wafer into individual electric/electronic elements to make the biocompatible electronic device.

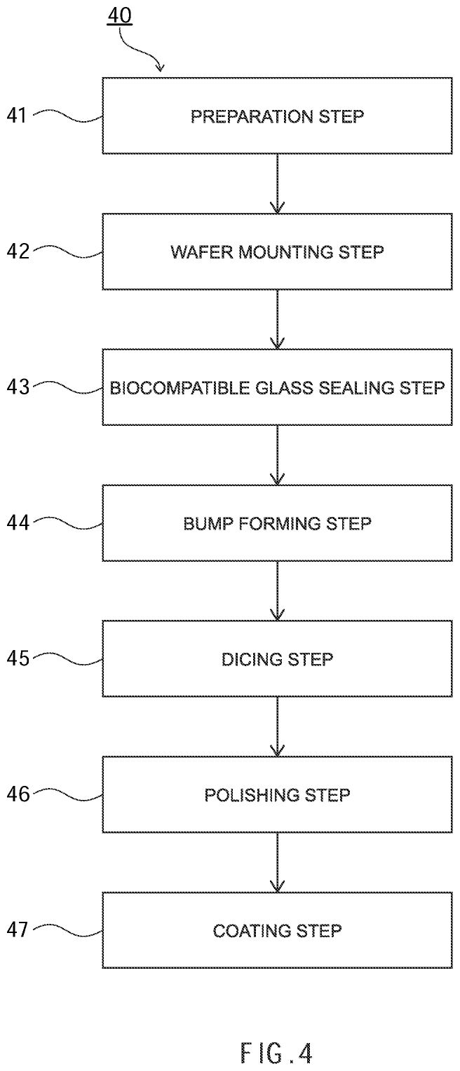

A manufacturing method 40 of a biocompatible electronic device of example 4 according to the present invention is a manufacturing method of the above described biocompatible electronic device 20. As illustrated in FIG. 4, the manufacturing method 40 of a biocompatible electronic device includes 1) a preparation step 41 of preparing a biocompatible glass substrate wafer with through electrode having a glass plate made of a biocompatible glass composed of only silicon, calcium, sodium, and oxygen that are elements existing in a body, including Na₂O and CaO in a base of SiO₂ as main components, and through electrodes made of a biocompatible metal provided by penetrating the glass plate and are made of platinum that is insoluble and noncorrosive in an environment in a body, and an Si semiconductor wafer to be used by being implanted in a living body, 2) a wafer mounting step 42 of butting desired electrodes of the biocompatible glass substrate wafer with through electrode and the Si semiconductor wafer to each other and bonding to the glass surface, 3) a biocompatible glass sealing step 43 of heating the mounted wafers in a furnace to hermetically seal a contact interface of the biocompatible glass and the Si semiconductor element and electrically connecting the mutual electrodes to form a glass sealed semiconductor wafer, 4) a bump forming step 44 of thereafter forming substantially spherical gold bumps on the through electrodes on another side of the biocompatible glass sealed semiconductor wafer, and 5) a dicing step 45 of dicing the biocompatible glass sealed semiconductor wafer after bump formation to separate the biocompatible glass sealed semiconductor wafer into individual electric/electronic elements to make the biocompatible electronic device. In the above described bump forming step 44, it is possible to mount a different semiconductor element from the above described Si semiconductor element, a passive components such as capacitors, resistors, coils, antennas, or sensors, and mechanism components onto the though electrodes in place of the gold bumps. Thereafter, a polishing step 46 of rounding corners of the biocompatible electronic device is performed to reduce mechanical irritations that are given to a living body. The polishing step 46 includes chemical and mechanical polishing to round corners of the glass surface. Furthermore, a coating step 47 of applying a biocompatible coating material composed of calcium hydroxide phosphate (C₁₀(PO₄)₆(OH)₂ of hydroxyapatite) to a surface of the biocompatible electronic device is performed as necessary. As for the coating, thin film coating of a thickness of 3 to 5 μm is applied by Chemical Vapour Deposition (CVD).

In the coating step 47 of applying the biocompatible coating material, it may be suitable to perform organic and inorganic composite coating by initially applying thin film coating of a thickness of 3 to 5 μm of an organic chemical material selected from nylon, polypropylene, polytetrafluoroethylene, and a polyamide resin to a surface of the biocompatible electronic device by the aforementioned CVD, and thereafter further applying a CVD coating film of the above described calcium hydroxide phosphate (Ca₁₀(PO₄)₆(OH)₂ of hydroxyapatite).

The biocompatible glass substrate with through electrode according to the present invention can directly hermetically seal a semiconductor element itself, and therefore contributes to making biocompatible electronic devices packageless and compact. Further, biocompatible electronic devices that are made flip-chip facilitate high density mounting and high functionality of the biocompatible electronic devices.

It should be considered that the embodiment disclosed this time is illustrative in all respects but is not restrictive. The scope of the present invention is not shown by the above described explanation, but shown by the claims, and intends to contain all changes within the meaning and the range equivalent to the claims.

The present invention can be used in a glass substrate with a through electrode for implant and an implant type electronic device.

### LIST OF REFERENCE SIGNS

10 biocompatible glass substrate with through electrode
11 glass plate
12 through electrode
20 biocompatible electronic device
21 glass plate
22 through electrode
23 biocompatible glass substrate with through electrodes
24 semiconductor element
25 bump
30 manufacturing method of biocompatible electronic device
31 preparation step
32 wafer mounting step
33 biocompatible glass sealing step
34 bump forming step
35 dicing step
40 manufacturing method of biocompatible electronic device
41 preparation step
42 wafer mounting step
43 biocompatible glass sealing step
44 bump forming step
45 dicing step
46 polishing step
47 coating step

1. A biocompatible glass substrate with a through electrode, comprising:
a glass plate made of a biocompatible glass; and
a through electrode made of a biocompatible metal provided by penetrating the glass plate.

2. The biocompatible glass substrate with through electrode according to claim 1, wherein at least a surface of the through electrode is made of a biocompatible metal.

3. The biocompatible glass substrate with through electrode according to claim 1, wherein the biocompatible glass is made of a glass composed of only silicon, boron, calcium, sodium, phosphorus, and oxygen that are elements existing in a body, including at least one of Na₂O and CaO in a base of SiO₂ as main components, and further including at least one of B₂O₃ and P₂O₃ as necessary.

4. The biocompatible glass substrate with through electrode according to claim 1, wherein the biocompatible metal comprises at least any one of platinum, tantalum, tungsten, titanium, a titanium alloy, a Co—Cr alloy, and stainless steel that are insoluble and noncorrosive in an environment in a body.

5. A biocompatible electronic device comprising a biocompatible glass substrate with through electrode having a glass plate made of a biocompatible glass, and a through electrode made of a biocompatible metal provided by penetrating the glass plate, and an electric/electronic device sealed onto the glass plate and electrically connected to the through electrode.

6. The biocompatible electronic device according to claim 5, wherein at least a surface of the through electrode is made of a biocompatible metal.

7. The biocompatible electronic device according to claim 5, wherein the biocompatible glass comprises a glass composed of only silicon, boron, calcium, sodium, phosphorus, and oxygen that are elements existing in a body, including at least one of Na₂O and CaO in a base of SiO₂ as main components, and further including at least one of B₂O₃ and P₂O₃ as necessary.

8. The biocompatible electronic device according to claim 5, wherein the biocompatible metal comprises at least any one of platinum, tantalum, tungsten, titanium, a titanium alloy, a Co—Cr alloy, and stainless steel that are insoluble and noncorrosive in an environment in a body.

9. The biocompatible electronic device according to claim 5, comprising a bump for connecting with the through electrode.

10. The biocompatible electronic device according to claim 9, wherein the bump comprises a biocompatible soft metal.

11. The biocompatible electronic device according to claim 10, wherein the soft metal is gold or a gold alloy.

12. The biocompatible electronic device according to claim 9, wherein any of an active component comprising a different semiconductor element from the electric/electronic device, a passive component comprising a capacitor, a resistor, a coil, an antenna or a sensor, or a mechanism component is mounted on the through electrode in place of some of the bumps.

13. The biocompatible electronic device according to claim 5, wherein biocompatible coating comprising an organic chemical material of nylon, polypropylene, polytetrafluoroethylene, or a polyamide resin, or an inorganic chemical material of calcium hydroxide phosphate, TiN, bioglass, carbon, alumina ceramics, or zirconia ceramics is further included on a surface of the device.

14. The biocompatible electronic device according to claim 13, wherein the biocompatible coating comprises organic and inorganic composite coating composed of the organic chemical material and the inorganic chemical material.

15. A manufacturing method of a biocompatible electronic device, comprising
1) a preparation step of preparing a biocompatible glass substrate wafer with through electrode including a glass plate of a biocompatible glass and through electrodes made of a biocompatible metal provided by penetrating the glass plate, and an electric/electronic device wafer,
2) a wafer mounting step of butting and bonding electrodes of the biocompatible glass substrate wafer with through electrode and the electric/electronic device wafer to each other,
3) a biocompatible glass sealing step of heating the mounted wafers in a furnace to hermetically seal a contact interface of the biocompatible glass and the electric/electronic device and electrically connecting the mutual electrodes to integrally form the biocompatible glass substrate wafer with through electrode and the electric/electronic device wafer,
4) a bump forming step of forming bumps for connection onto the through electrodes of the wafer integrally formed, after the biocompatible glass sealing step, and
5) a dicing step of dicing the integrally formed wafer after bump formation, to separate the wafer into individual electric/electronic elements to make the biocompatible electronic device.

16. The manufacturing method of a biocompatible electronic device according to claim 15, wherein in the through electrodes, at least surfaces are made of the biocompatible metal.

17. The manufacturing method of a biocompatible electronic device according to claim 15, wherein the biocompatible glass comprises a glass that is composed of only silicon, boron, calcium, sodium, phosphorus, and oxygen that are elements existing in a body, including at least one of Na₂O and CaO in a base of SiO₂ as main components, and further including at least one of B₂O₃ and P₂O₃ as necessary.

18. The manufacturing method of a biocompatible electronic device according to claim 15, wherein the biocompatible metal comprises any one of platinum, tantalum, tungsten, titanium, a titanium alloy, a Co—Cr alloy, and stainless steel that are insoluble and noncorrosive in an environment in a body.

19. The manufacturing method of a biocompatible electronic device according to claim 15, wherein the bump comprises a biocompatible soft metal.

20. The manufacturing method of a biocompatible electronic device according to claim 19, wherein the soft metal is gold or a gold alloy.

21. The manufacturing method of a biocompatible electronic device according to claim 15, wherein after the dicing step, a polishing step of rounding corners of a biocompatible electronic device is added to reduce a mechanical irritation to be given to a living body.

22. The manufacturing method of a biocompatible electronic device according to claim 15, wherein after the dicing step, a coating step of applying on a surface of the device a biocompatible coating material comprising an organic chemical material of nylon, polypropylene, polytetrafluoroethylene, and a polyamide resin, or an inorganic chemical material of calcium hydroxide phosphate, TiN, bioglass, carbon, alumina ceramics or zirconia ceramics is added.

23. The manufacturing method of a biocompatible electronic device according to claim 22, wherein the biocompatible coating material comprises organic and inorganic composite coating composed of the organic chemical material and the inorganic chemical material.

24. The manufacturing method of a biocompatible electronic device according to claim 22, wherein the biocompatible coating material is thin film coating of a thickness of approximately 2 to 30 μm applied by Chemical Vapour Deposition (CVD).

25. The manufacturing method of a biocompatible electronic device according to claim 15 characterized in that in the bump forming step, any of an active component comprising a different semiconductor element from the electric/electronic device, a passive component comprising a capacitor, a resistor, a coil, an antenna or a sensor or a mechanism component is mounted on the through electrodes, in place of some of the bumps.
